(19) United States
(12) Patent Application Publication
(10) Pub. No.: US 2025/0221648 A1
(43) Pub. Date: Jul. 10, 2025

(54) SYSTEM FOR TAKING AND EVALUATING ELECTROCARDIOGRAMS USING DRY ELECTRODES

(71) Applicant: HelpWear Inc., Toronto (CA)

(72) Inventors: Andre Bertram, Toronto (CA); Derek Exner, Calgary (CA); Owen Gillett, Toronto (CA)

(21) Appl. No.: 19/013,756

(22) Filed: Jan. 8, 2025

Related U.S. Application Data
(60) Provisional application No. 63/618,512, filed on Jan. 8, 2024.

(51) Int. Cl. A61B 5/282; A61B 5/00; A61B 5/0205; A61B 5/11; A61B 5/256; A61B 5/276; A61B 5/308; A61B 5/332; A61B 5/339; A61B 5/35

(52) U.S. Cl. CPC A61B 5/282; A61B 5/0006; A61B 5/0205; A61B 5/256; A61B 5/276; A61B 5/308; A61B 5/332; A61B 5/339; A61B 5/35; A61B 5/6841; A61B 5/7203; A61B 5/7221; A61B 5/1118; A61B 2562/0209

(57) ABSTRACT

Disclosed herein is a cardiac monitoring system comprising a monitoring device comprising a plurality of electrodes and one or more processors. The one or more processors are configured to, when the monitoring device is placed around an extremity of a patient, control the plurality of electrodes to record one or more electrocardiogram signals. The one or more processors are further configured to receive, from the monitoring device, one or more data packets, the one or more data packets indicative of the one or more electrocardiogram signals. The one or more processors are also configured to apply one or more adaptive software filters to the one or more data packets to extract an electrocardiogram for the patient. The one or more processors are also configured to analyze the extracted electrocardiograms using one or more cardiac detection tools to identify abnormal electrocardiograms and output the identified abnormal electrocardiograms.

### CROSS-REFERENCE TO RELATED APPLICATION(S)

This application claims the benefit under 35 U.S.C. § 119 (e) to U.S. Provisional Application 63/618,512, filed Jan. 8, 2024 and entitled “SYSTEM FOR TAKING AND EVALUATING ELECTROCARDIOGRAMS USING DRY ELECTRODES,” which is hereby incorporated herein by reference in its entirety.

### FIELD

The various examples herein relate to capturing and evaluating electrocardiograms.

### BACKGROUND

An electrocardiogram (ECG) is a simple test used to evaluate the rate and rhythm of the heart of a patient. Electrodes, typically in the form of small, plastic patches containing metallic sensors that use adhesive to stick to the skin of the patient, are placed at certain spots on the chest, arms, and legs. The electrodes are connected to an ECG machine by lead wires. The electrical activity of the heart is then measured, interpreted, and printed out.

Natural electrical impulses coordinate contractions of the different parts of the heart to keep blood flowing the way it should. An ECG records these impulses to show how fast the heart is beating, the rhythm of the heart beats (steady or irregular), and the strength and timing of the electrical impulses as they move through the different parts of the heart. Changes in an ECG can be a sign of many heart-related conditions.

While the test is simple, current ECG procedures can cause issues for the patients. Many patients have negative reactions to the adhesive used to stick the electrodes to the skin of the patient, leading to allergic reactions, irritated skin, pulled hair, and painful removal of the electrodes.

### BRIEF SUMMARY

Discussed herein are various systems and methods for taking and evaluating electrocardiograms for patients using certain electrodes that do not require gel or other materials designed to enhance electrical conduction or remove air between the electrodes and the skin of the patient being evaluated. A computing device controls a plurality of dry electrodes on a cardiac monitoring device to record one or more electrocardiogram signals when placed around an extremity of a patient. The computing device receives the one or more electrocardiogram signals from the cardiac monitoring device. The computing device applies one or more adaptive software filters to the one or more data packets to extract an electrocardiogram for the patient.

The techniques of this disclosure may further include evaluation tools for the electrocardiograms. A computing device analyzes a plurality of electrocardiograms for a patient, each electrocardiogram of the plurality of electrocardiograms being recorded at unique times. The computing device determines that a subset of electrocardiograms from the plurality of electrocardiograms include a rhythmic abnormality. The computing device generates a graphical user interface that includes at least a first grouping of graphical indications and a second grouping of one or more graphical indications, each graphical indication in the first grouping representing an electrocardiogram from the subset of electrocardiograms that include the rhythmic abnormality, and each graphical indication in the second grouping representing an electrocardiogram from the plurality of electrocardiograms that is not included in the subset of electrocardiograms. The computing device outputs, for display on a display device, the graphical user interface.

The cardiac monitoring system as described herein may be a prescription-quality solution designed to facilitate end-to-end ambulatory ECG recording, signal processing, and data reporting. The system is intended to either to diagnose patients' suspected cardiac arrhythmias or to monitor the frequency (burden) of patients' known arrhythmias over time.

Unlike other ambulatory ECG monitors available today, the electrodes on the system strap/main unit do not need conductive gel to enable high-def signal acquisition. Not requiring gel means that no adhesives are needed as part of the system design. Other ambulatory ECG monitors require strong adhesives to encapsulate their electrode gel and hold the devices in place. Which therefore restricts the wear time of those products to 14-30 days before the onset of skin irritation or skin erosion (necrosis).

Without adhesives or gels, the system makes it possible to target a different site of monitoring (e.g., the upper bicep or some other extremity or body part), in contrast to known monitors, which are typically positioned on the torso. This location has proven to be both comfortable and unobtrusive for the patient, allowing the system examples herein to theoretically be worn in perpetuity. It is envisioned to be prescribed for multiple months to optimize the likelihood that a future arrhythmia event may be recorded for a physician to leverage as part of a patient's adjusted treatment plan.

In Example 1, a cardiac monitoring system comprises (a) a monitoring device comprising a plurality of electrodes; and (b) one or more processors configured to: (i) when the monitoring device is placed around an extremity of a patient, control the plurality of electrodes to record one or more electrocardiogram signals; (ii) receive, from the monitoring device, one or more data packets, the one or more data packets indicative of the one or more electrocardiogram signals; and (iii) apply one or more adaptive software filters to the one or more data packets to extract an electrocardiogram for the patient.

Example 2 relates to the cardiac monitoring system according to Example 1, wherein the plurality of electrodes of the monitoring device comprises a plurality of dry electrodes that, when placed around the extremity of the patient, do not require conductive gel and do not require adhesive to record the one or more electrocardiogram signals.

Example 3 relates to the cardiac monitoring system according to any one or more of Examples 1-2, wherein each of the plurality of electrodes comprises a high-sensitivity analog front end circuit.

Example 4 relates to the cardiac monitoring system of Example 3, wherein each of the high-sensitivity analog front end circuits comprises one or more of a right leg drive (RLD) feedback system and a direct current (DC) biasing reduction system.

Example 5 relates to the cardiac monitoring system of any one or more of Examples 3-4, wherein, while the plurality of electrodes are recording the one or more electrocardiogram signals, the high-sensitivity analog front end circuits are configured to reduce noise in the one or more electrocardiogram signals, wherein the one or more data packets are indicative of the one or more electrocardiogram signals after noise reduction.

Example 6 relates to the cardiac monitoring system of Example 5, wherein the high-sensitivity analog front end circuits being configured to reduce the noise in the one or more electrocardiogram signals comprises the high-sensitivity analog front end circuits being configured to: (i) amplify the one or more electrocardiogram signals; and (ii) apply one or more of an RLD feedback system, a DC biasing reduction system, and one or more analog filters to the one or more electrocardiogram signals to reduce the noise in the one or more electrocardiogram signals, wherein the one or more analog filters are either fixed analog filters, adaptive analog filters, or a combination of fixed and adaptive analog filters.

Example 7 relates to the cardiac monitoring system of any one or more of Examples 1-6, wherein the one or more processors are further configured to: (i) analyze the electrocardiogram of the patient; and (ii) detect one or more rhythm abnormalities in the electrocardiogram.

Example 8 relates to the cardiac monitoring system of Example 7, wherein each of the one or more rhythm abnormalities corresponds to a section of the electrocardiogram, wherein the one or more processors are further configured to generate a graphical user interface that presents at least the distinct, non-consecutive QRS wave sections of the electrocardiogram,

Example 9 relates to the cardiac monitoring system of any one or more of Examples 1-9, wherein the one or more processors are further configured to: (i) generate a graphical user interface that includes an indication of the electrocardiogram; and (ii) output, for display on a display device, the graphical user interface.

Example 10 relates to the cardiac monitoring system of any one or more of Examples 1-9, wherein the one or more processors are further configured to: (i) receive, from one or more ancillary devices, activity information; (ii) generate a graphical user interface that includes an indication of the electrocardiogram and an indication of at least a portion of the activity information; and (iii) output, for display on a display device, the graphical user interface.

Example 11 relates to the cardiac monitoring system of Example 10, wherein the activity information comprises one or more of a daily step count, an activity level, a sleep pattern, a blood pressure, a pulse oxygenation level, a carbon dioxide level, a detected fall, sleep apnea detection, a hemodynamic measurement, a skin color, a skin temperature, a sweat detection, and posture detection.

Example 12 relates to the cardiac monitoring system of any one or more Examples 1-11, wherein the one or more processors are further configured to: (i) monitor a one or more biometrics of the user, the one or more metrics comprising one or more of a blood pressure for the user, fall detection, a respiratory rate for the user, a measure of hemodynamic flow for the user, skin pigmentation level of the user, a skin temperature for the user, and galvanic sweat levels for the user; (ii) generate a graphical user interface that includes an indication of the one or more biometrics and the electrocardiogram; and (iii) output, for display on a display device, the graphical user interface.

Example 13 relates to the cardiac monitoring system of any one or more Examples 1-12, wherein the one or more processors are further configured to: (i) determine one or more of a respiratory rate for the user, a heart rate for the user, and motion of the user; and (ii) combine one or more of the respiratory rate, the heart rate, and the motion during sleep to detect sleep apnea.

Example 14 relates to the cardiac monitoring system of any one or more of Examples 1-13, wherein the one or more processors are part of a computing device, wherein the computing device comprises one of a mobile computing device, a cloud-based server, or a desktop computing device.

Example 15 relates to the cardiac monitoring system of any one or more of Examples 1-14, wherein the one or more processors are part of a computing device, wherein the computing device comprises a distributed computing system comprising two or more of a mobile computing device, a cloud-based server, or a desktop computing device.

Example 16 relates to the cardiac monitoring system of any one or more of Examples 1-15, wherein the one or more processors are further configured to: (i) analyze the electrocardiogram of the patient; and (ii) diagnose a condition in the patient based on the electrocardiogram.

Example 17 relates to the cardiac monitoring system of any one or more of Examples 1-16, wherein the one or more processors are further configured to: (i) receive an indication of user input comprising a human annotation of detected rhythm abnormalities to generate an annotated electrocardiogram; and (ii) transmit the annotated electrocardiogram to a computing device associated with a prescribing physician.

Example 18 relates to the cardiac monitoring system of any one or more of Examples 1-17, wherein the one or more processors are further configured to: (i) repeat the steps in Example 1 to extract a plurality of electrocardiograms for the patient, each cardiogram being associated with a unique time span for the patient; and (i) determine one or more of a frequency and a pattern of arrhythmias across the plurality of electrocardiograms for the patient.

Example 19 relates to the cardiac monitoring system of any one or more of Examples 1-18, wherein the monitoring device comprises a strap, the strap comprises one or more of: (a) a hook-and-loop strap, (b) a buckle clip strap, (c) a belt strap, (d) an elastic loop, and (e) an elastomer strap with one or more of the plurality of electrodes embedded within the elastomer strap.

Example 20 relates to the cardiac monitoring system of any one or more of Examples 1-19, wherein the extremity of the patient comprises an arm of the patient or a leg of the patient.

Example 21 relates to the cardiac monitoring system of any one or more of Examples 1-20, wherein the one or more electrocardiogram signals comprise one or more clinically relevant electrocardiogram signals.

In Example 22, a cardiac monitoring device comprises (a) a self-adhering adjustable length strap; and (b) a plurality of electrodes configured to record one or more electrocardiogram signals when the cardiac monitoring device is placed around an extremity of a patient, wherein the cardiac monitoring device, when placed around the extremity of the patient, does not require conductive gel and does not require adhesive to record the one or more electrocardiogram signals.

Example 23 relates to the cardiac monitoring device of Example 22, wherein each of the plurality of electrodes comprises a high-sensitivity analog front end circuit.

Example 24 relates to the cardiac monitoring device of Example 23, wherein each of the high-sensitivity analog front end circuits comprises one or more of a common mode noise reduction system, a right leg drive (RLD) feedback system, and a direct current (DC) biasing reduction system.

Example 25 relates to the cardiac monitoring device of any one or more of Examples 22-24, wherein, while the plurality of electrodes are recording the one or more electrocardiogram signals, the high-sensitivity analog front end circuits are configured to reduce noise in the one or more electrocardiogram signals, wherein the one or more data packets are indicative of the one or more electrocardiogram signals after noise reduction.

Example 26 relates to the cardiac monitoring device of any one or more of Examples 22-25, wherein the strap comprises one or more of: (a) a hook-and-loop strap, (b) a buckle clip strap, (c) a belt strap, (d) an elastic loop, and (e) an elastomer strap with one or more of the plurality of electrodes embedded within the elastomer strap.

Example 27 relates to the cardiac monitoring device of any one or more of Examples 22-26, wherein the extremity of the patient comprises an arm of the patient or a leg of the patient.

In Example 28, a method comprises (i) controlling, by one or more processors, a plurality of dry electrodes on a cardiac monitoring device to record one or more electrocardiogram signals when placed around an extremity of a patient; (ii) receiving, by the one or more processors, the one or more electrocardiogram signals from the cardiac monitoring device; and (iii) apply one or more adaptive software filters to the one or more data packets to extract an electrocardiogram for the patient.

Example 29 relates to the method of Example 28, further comprising training an electrocardiogram model, the electrocardiogram model comprising a plurality of patient specific templates on prior clean data.

Example 30 relates to the method of Example 29, wherein the plurality of patient specific templates each comprise average PQRST models.

Example 31 relates to the method of any one or more of Examples 28-30, wherein applying the one or more adaptive filters comprises applying the electrocardiogram model to the one or more electrocardiogram signals to remove one or more distortions in the one or more electrocardiogram signals to extract the electrocardiogram for the patient.

Example 32 relates to the method of any one or more of Examples 28-31, wherein the one or more electrocardiogram signals comprise a first set of electrocardiogram signals, and wherein the method further comprises: (i) controlling, by the one or more processors, the plurality of dry electrodes to record a second set of electrocardiogram signals; (ii) receiving, by the one or more processors, the second set of electrocardiogram signals from the cardiac monitoring device; (iii) analyzing, by the one or more processors, the second set of electrocardiogram signals; (iv) determining, by the one or more processors, an error in the recording of the second set of electrocardiogram signals; and (v) outputting, by the one or more processors, an alert indicating the error in the recording of the second set of electrocardiogram signals.

Example 33 relates to the method of Example 32, wherein the error in the recording of the second set of electrocardiogram signals comprises one or more of a poor signal quality, a device-off-body detection error, and a poor positioning of the cardiac monitoring device.

Example 34 relates to the method of Example 33, wherein the error comprises the poor positioning of the cardiac monitoring device, wherein the alert comprises a template guided positioning tool.

Example 35 relates to the method of Example 32, further comprising, in response to determining the error: (i) controlling, by the one or more processors, the plurality of dry electrodes to record a third set of electrocardiogram signals; (ii) receiving, by the one or more processors, the third set of electrocardiogram signals from the cardiac monitoring device; (iii) processing, by the one or more processors, a set duration of time within the third set of electrocardiogram signals; (iv) outputting, by the one or more processors and to a clinical display device, the processed third set of electrocardiogram signals over the set duration of time for review by an operator.

Example 36 relates to the method of any one or more of Examples 28-34, wherein the one or more adaptive filters comprise one or more of an initial pass rhythm classification tool, a QRS detection algorithm, a peak finder detection algorithm, a physiological constraint model, and a patient specific template.

In Example 37, a method comprises (i) analyzing, by one or more processors, a plurality of electrocardiograms for a patient, each electrocardiogram of the plurality of electrocardiograms being recorded at unique times; (ii) determining, by the one or more processors, that a subset of electrocardiograms from the plurality of electrocardiograms include a rhythmic abnormality; (iii) generating, by the one or more processors, a graphical user interface that includes at least a first grouping of graphical indications and a second grouping of one or more graphical indications, each graphical indication in the first grouping representing an electrocardiogram from the subset of electrocardiograms that include the rhythmic abnormality, and each graphical indication in the second grouping representing an electrocardiogram from the plurality of electrocardiograms that is not included in the subset of electrocardiograms; and (iv) outputting, by the one or more processors and for display on a display device, the graphical user interface.

Example 38 relates to the method of Example 37, further comprising generating, by the one or more processors, the graphical user interface to include an overlay of an averaged template beat for the patient with each graphical indication in the first grouping.

Example 39 relates to the method of any one or more of Examples 37-38, further comprising: (i) determining, by the one or more processors, a diagnostic recommendation based on the subset of electrocardiograms that include a rhythmic abnormality; and (ii) generating, by the one or more processors, the graphical user interface to further include a graphical indication of the diagnostic recommendation.

Example 40 relates to the method of any one or more of Examples 37-39, further comprising: (i) automatically generating, by the one or more processors, one or more clinical notes based on the subset of electrocardiograms that include a rhythmic abnormality; and (ii) generating, by the one or more processors, the graphical user interface to further include a graphical indication of the one or more clinical notes.

In Example 41, a method comprises steps performed by the cardiac monitoring system of any one or more of Examples 1-21, the cardiac monitoring device of any one or more of Examples 22-27, or the methods of any one or more of Examples 28-40.

In Example 42, a non-transitory computer-readable storage medium comprises instructions that, when executed, cause one or more processors of a computing device to perform the steps performed by the cardiac monitoring system of any one or more of Examples 1-21, the cardiac monitoring device of any one or more of Examples 22-27, or the methods of any one or more of Examples 28-40.

In Example 43, the Example includes any technique described herein.

While multiple examples are disclosed, still other examples will become apparent to those skilled in the art from the following detailed description, which shows and describes illustrative examples. As will be realized, the various implementations are capable of modifications in various obvious aspects, all without departing from the spirit and scope thereof. Accordingly, the drawings and detailed description are to be regarded as illustrative in nature and not restrictive.

### BRIEF DESCRIPTION OF THE DRAWINGS

The following drawings are illustrative of particular examples of the present disclosure and therefore do not limit the scope of the invention. The drawings are not necessarily to scale, though examples can include the scale illustrated, and are intended for use in conjunction with the explanations in the following detailed description wherein like reference characters denote like elements. Examples of the present disclosure will hereinafter be described in conjunction with the appended drawings.

FIG. 1 is a diagram illustrating a cardiac monitoring device in communication with a computing device that may be separate from or integrated into the cardiac monitoring device, in accordance with the techniques of this disclosure.

FIG. 2 is a block diagram illustrating a more detailed example of a computing device configured to perform the techniques described herein.

FIG. 3 is a diagram illustrating an example cardiac monitoring device, in accordance with the techniques of this disclosure.

FIG. 4 is an example user interface on a client device showing data from electrocardiograms, in accordance with the techniques of this disclosure.

FIG. 5 is an example flow diagram illustrating measuring electrocardiogram signals from a cardiac monitoring device and transmitting this data to a certified technologist, in accordance with the techniques of this disclosure.

FIG. 6 is a flow diagram illustrating an example method for extracting an electrocardiogram from signals recorded by a cardiac monitoring device, in accordance with the techniques of this disclosure.

FIG. 7 is a flow diagram illustrating an example method for analyzing one or more electrocardiograms extracted from signals recorded by a cardiac monitoring device, in accordance with the techniques of this disclosure.

### DETAILED DESCRIPTION

The following detailed description is exemplary in nature and is not intended to limit the scope, applicability, or configuration of the techniques or systems described herein in any way. Rather, the following description provides some practical illustrations for implementing examples of the techniques or systems described herein. Those skilled in the art will recognize that many of the noted examples have a variety of suitable alternatives.

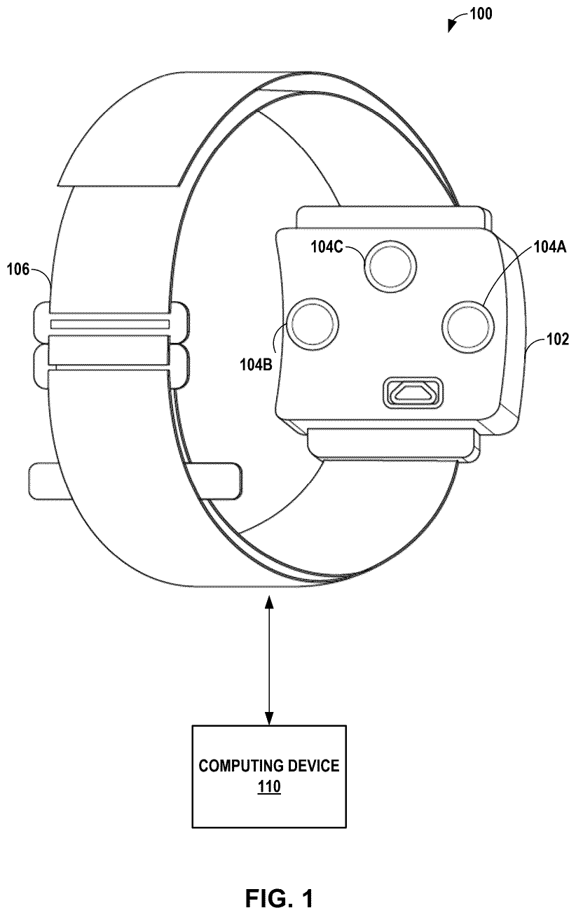

FIG. 1 is a diagram illustrating system 100 that includes cardiac monitoring device 102 in communication with computing device 110 that may be separate from or integrated into cardiac monitoring device 102, in accordance with the techniques of this disclosure.

Cardiac monitoring device 102 may include a strap 106 such that cardiac monitoring device 102 may be wrapped securely around an extremity of a patient, like an arm or a leg. Strap 106 may be any strap that can secure cardiac monitoring device 102 to the extremity of the patient, such as elastic, nylon, silicone, or any other sturdy and flexible material, with a securing mechanism (e.g. elasticity, belt buckle, clip buckle, etc.). Cardiac monitoring device 102 may include one or more dry electrodes, such as electrodes 104A, 104B, and 104C. Dry electrodes 104A-104C may be placed directly on the skin of a patient without requiring gel or other material designed to enhance electrical conduction or reduce or eliminate the air existing between the electrode and the skin of the patient. In some instances, dry electrodes 104A-104C may each be pairs or larger groups of dry electrodes, raising the number of dry electrodes in cardiac monitoring device 102 to four or more dry electrodes. In some instances, such as when strap 106 is made of an elastomer, some sensors, such as any of dry electrodes 104A-104C or additional sensors for measuring the biometric signals of a patient for constructing an ECG, may be embedded within the elastomer material of strap 106 rather than in a separate component attached to strap 106.

Computing device 110 may be any computer with the processing power required to adequately execute the techniques described herein. Computing device 110 may be incorporated into cardiac monitoring device 102 or separate from (and in wired or wireless communication with) cardiac monitoring device 102. For instance, computing device 110 may be any one or more of a mobile computing device (e.g., a smartphone, a tablet computer, a laptop computer, etc.), a desktop computer, a smarthome component (e.g., a computerized appliance, a home security system, a control panel for home components, a lighting system, a smart power outlet, etc.), a vehicle, a wearable computing device (e.g., a smart watch, computerized glasses, a heart monitor, a glucose monitor, smart headphones, etc.), a virtual reality/augmented reality/extended reality (VR/AR/XR) system, a video game or streaming system, a transponder, a network modem, router, or server system, or any other computerized device that may be configured to perform the techniques described herein.

In accordance with the techniques of this disclosure, various systems and methods are described for taking and evaluating electrocardiograms for patients using certain electrodes that do not require gel or other materials designed to enhance electrical conduction or remove air between the electrodes and the skin of the patient being evaluated. Computing device 110 controls a plurality of dry electrodes 104A-104C on cardiac monitoring device 102 to record one or more electrocardiogram signals when placed around an extremity of a patient. Computing device 110 receives the one or more electrocardiogram signals from cardiac monitoring device 102. Computing device 110 applies one or more adaptive software filters to the one or more data packets to extract an electrocardiogram for the patient.

The cardiac monitoring system as described herein may be a prescription-quality solution designed to facilitate end-to-end ambulatory ECG recording, signal processing, and data reporting. The system is intended to either to diagnose patients' suspected cardiac arrhythmias or to monitor the frequency (burden) of patients' known arrhythmias over time.

Unlike other available ambulatory ECG monitors, the electrodes on the system strap/main unit do not need conductive gel to enable high-def signal acquisition. Not requiring gel means that no adhesives are needed as part of the system design. Other EKG monitors require strong adhesives to encapsulate their electrode gel and hold the devices in place. Which therefore restricts the wear time of those products to 14-30 days before the onset of skin irritation or skin erosion (necrosis).

Without adhesives or gels, the system makes it possible to target a different site of monitoring (upper bicep), in contrast to known monitors, which are typically positioned on the torso. This location has proven to be both comfortable and unobtrusive for the patient, allowing the system examples herein to theoretically be worn in perpetuity. It is envisioned to be prescribed for multiple months to optimize the likelihood that a future arrhythmia event may be recorded for a physician to leverage as part of a patient's adjusted treatment plan.

Conventional non-invasive ECG monitoring systems utilize wet conductive-gel electrodes worn on a patient's chest to collect, store and report on a patient's heart health. Due to the damage to the patient's skin, these devices are limited to a wear time of 30 days. The system described herein provides an end-to-end ambulatory ECG collecting, processing and reporting solution to record medium to long term monitoring sessions. Enabled by dry electrode technology mechanically fixed to a different target application site on the patient's extremity, like a leg or an arm, the system described herein can provide continuous ECG monitoring for periods of 24 hours to theoretically be worn in perpetuity. In doing so, the system described herein is capable of extending the length of clinical ECG monitoring and is intended to either diagnose patients' suspected cardiac arrhythmias (frequent or infrequent) or to monitor the frequency (burden) of the patients' known arrhythmias over time.

The system described herein utilizes dry electrode technology to capture raw ECG signals from the patient's arm that is captured by an analog front end circuit. In examples where four electrodes are used, the four electrode system encompasses a traditional two electrode system to capture the electrical dipole created by the heart while pumping blood around the body. Additionally, the system described herein includes two unconventional electrodes to reduce the noise captured in the signal. These four inputs are processed through an analog front-end circuit that outputs a clean clinically relevant ECG signal. The system communicates via Bluetooth to a user's smartphone device using a mobile application to then send the electrocardiogram signal to a cloud system. The cloud system receives the data, runs the data through smoothing algorithms and traditional and/or ML based analytic tools and stores both the complete dataset and any suspected cardiac arrhythmias detected. Finally, healthcare professionals are able to access this data in the form of a summary report, review it and provide a final diagnosis on the patient's heart health. The technology's target location on a patient's extremity is a core technological improvement that is enabled by the following features: Dry electrode technology, an analog front end circuit design, mechanical enclosure and strap to affix the device to the patient's arm (with little noise injection), software data filtering methods, traditional and machine learning (ML) derived suspected cardiac arrhythmia detectors and rhythm identification tools, user guided software tools to facilitate high quality data acquisition, and supplementary sensors to inform cardiac health diagnostics (including but not limited to SPO2 accelerometer, gyroscope, skin temperature, hemodynamic sensor, skin color, and galvanic sweat levels.)

The system described herein is an improvement on traditional ECG monitoring technology. The system analog circuit is built on the fundamentals of ECG monitoring. The principle records an electrical potential across the heart's muscles using a positive and negative terminal electrode on opposite sides of the heart. The two terminals measure a dipole across the heart and typically are placed on the chest. These potentials are differentiated to produce an electrical projection of the heart's health. The system expands on the traditional technology in applying this principle while placing the technology on to the patient's left arm. The two electrodes on the arm collect these electrical potentials via vectors in similar directions. This creates the challenge of recording a clinically relevant ECG signal as the signal amplitudes collected are two scales of magnitude smaller. To collect a clinically relevant signal, the technology implements noise reduction methods, namely a DC voltage biasing electrode and right leg driven circuit. In addition, the technology's mechanical features and digital signal processing are optimized to overcome the exaggerated challenges of single arm ECG monitoring.

The system described herein also incorporates existing methods to transfer the collected ECG signal through the system described herein from the device to the phone to cloud services.

The system described herein is a prescription-quality solution designed to facilitate end-to-end ambulatory ECG recording, signal processing, and data reporting. The system is intended to either diagnose patients' suspected cardiac arrhythmias or to monitor the frequency (burden) of patients' known arrhythmias over time.

The system described herein may include: patient-worn hardware (e.g., a bicep-worn strap with a plurality of metallic electrodes, such as four or more metallic electrodes, to record clinically-relevant high-definition ECG signals); a user-associated computing device executing software (e.g., a mobile phone executing a phone-based mobile application to facilitate the transfer of the patient's ECG data to a secure cloud server; a server device (a cloud-based processor and algorithm stack to process the ECG signals and make first-pass arrhythmia classifications and diagnoses); and software (e.g., desktop software to facilitate human annotation of detected rhythm abnormalities by certified cardiac technologists).

The mobile application may also (eventually) display ancillary information to the patients, such as their daily step count, activity level, sleep patterns, SPO2 levels, skin temperature, skin color, and galvanic sweat levels. The software may enable these technologists to easily create rhythm reports to be viewed by the prescribing physician.

Unlike other ambulatory ECG monitors available today, the electrodes on the cardiac monitoring device described herein do not need conductive gel to enable high-definition signal acquisition. Not requiring gel means that no adhesives are needed as part of the design of the system described herein. Other EKG/ECG monitors require strong adhesives to encapsulate their electrode gel and hold the devices in place, which therefore restricts the wear time of those products to 14-30 days before the onset of skin irritation or skin erosion (e.g., necrosis).

Without the use of adhesives or gels, the system described herein has therefore been able to target an atypical site of monitoring (e.g., upper bicep). This location has proven to be both comfortable and unobtrusive for the patient, allowing a cardiac monitoring device as described herein to theoretically be worn in perpetuity—but is envisioned to be prescribed for multiple months to optimize the likelihood that a future arrhythmia event may be recorded for a physician to leverage as part of a patient's adjusted treatment plan.

This dry-electrode solution is enabled by a highly sensitive analog front-end circuit which has been designed to extract the highly attenuated signals collected by the system. Collecting ECG signals from the patient's arm poses a challenge of detecting a largely attenuated signal. This is due to the signal attenuation that occurs while the signal travels through the patient's tissue as it reaches the arm and the narrow inter-electrode distance of an ambulatory ECG monitor on a patient's extremity (e.g., arm). To overcome this, the front-end circuit may have a high impedance input buffer operation amplifier. In addition, the circuit refined its performance by implementing two noise reduction systems-a Right Leg Drive (RLD) feedback system (to remove common mode noise) and a DC biasing reduction system to remove the unwanted DC offset.

Collecting ECG using dry electrodes from the user's arm poses other challenges that require the use of aggressive hardware and software filters. Due to the device's proximity to the patient's bicep, muscle and motion artifacts are more prominent than a standard chest based ECG monitor. High order hardware filters are utilized within the system described herein to more aggressively remove all frequencies outside of the desired bandpass frequency range, such as a range of 0.67-40 Hz (although other ranges with different bounds could also be utilized and are also contemplated for the purposes of this disclosure).

The system described herein may be expanded with the implementation of multiple electrodes around the patient's arm to capture multiple electrical dipoles (ECG leads). The use of multiple leads (from 1 to n) provides the ability to select an optimal single lead that is least contaminated by noise furthering the quality of the final ECG signal provided to clinicians. In one example, a single frontend circuit may be used, of which a multiplexing circuit may be digitally controlled to switch between leads, with an on-board processor, to select the optimal lead to be transferred to cloud services. In a different example, the entire analog frontend may be replicated to capture in parallel, n leads, in which all leads may be recorded, processed and reported to the clinicians. In doing so, the system described herein can provide different electrical dipoles (images) of the heart's cardiac function to lead to a more informed clinical diagnoses.

The skin-electrode interface in biometric sensing influences the quality of the collected signal. Other ECG monitoring systems found on the chest use wet electrodes that struggle to adhere to a patient's body for longer than 30 days, as the wet electrodes' adhesives wear down and sweat builds between the skin and electrode. As the electrode adhesives wear down, the quality of the signal degrades. The target location of the system described herein permits the use of a self-adjusting elastic strap design to affix the cardiac monitoring device to the patient. This system is optimized to remain tight to the patient's arm. It does so by stretching and contracting as the circumference of the patient's arm changes in size during muscle activation and movement, reducing the amount of recorded motion artifact. To further the system's resistance to motion artifacts, the main components of the system are housed with two separate rigid enclosures with a flexible bridge between them. This design permits the two shells to move independent of each other during arm motion, reducing the generated motion artifact noise's impact on the electrodes within the skin-electrode interface. These two design systems can accommodate a range of arm sizes and shapes and meet the market's needs.

The cardiac monitoring device described herein contains a single (or multiple) electrode(s) that is affixed to the strap using a semi-rigid base. The semi-rigid base can flex while the patient's arm is in motion to maintain a strong adhesion to the patient's arm, providing both a comfortable wear and a high quality ECG signal. An additional feature of the strap design may be the adjustable/self-closing zipper rail. The system permits the user to slide the sensor along the strap with ease while enclosing all the sensor technology within the strap.

The system described herein was built with removability in mind as the desired use of the technology can be for prolonged periods where the removal of the system for showering/washing the device is typically observed. A quick release mechanism was invented to expand the circumference of the strap quickly to allow the strap to slide up/down the patient's arm. The quick release utilizes a configuration of loops in conjunction with a magnet to hold the strap in place while on the patient's arm. The design loops auto lock in position while the magnet is engaged due to the increased friction that the straps force on the patient's arm. Once disengaged, the friction between the patient's arm and strap is released, permitting an easy removal of the strap.

To further overcome the challenges described above when recording ECG signals from a patient's arm, an adaptive aggressive software filtering is used. This tool utilizes patient specific templates (average PQRST models) generated on prior clean data to extract and remove distortions in the current ECG signal to provide a clear final output. The filter adaptively uses more aggressive smoothing techniques depending on the measured amount of in range frequency noise (e.g., frequencies >30 Hz and <5 Hz, although other bounds could also be utilized and are also contemplated for the purposes of this disclosure) present.

The system described herein provides an end-to-end ECG monitoring solution with data processing and rhythm classification tools to help improve the clinically interpretability of the system. Similar to other ECG monitoring solutions, the system described herein detects all clinically relevant heart arrhythmias and reports these events to clinicians for their final review to diagnose the patient's health. The software's rhythm classification tools are unique in its application due to the system collecting ECG data with dry electrodes from a patient's single arm. The rhythm classification tools begin with a robust QRS detection algorithm. The tool works by applying a narrow frequency band filter upon the data to extract and amplify QRS-like peaks. A robust peak finder detection algorithm is then used by thresholding the manipulated signal. Next, physiological constraints are placed on the detected QRS complexes to remove suspected false positives. Then exploiting the ECG's periodic nature, the above mentioned patient specific template feature is slid over the original signal looking for matching signatures between all templates and each individual beat, removing suspected false positives.

The rhythm classification tools use the above mentioned QRS detections system and data filtered system to classify beats and rhythms. The tool uses a multitude of features including but not limited to; QRS complex width, R-R interval, P wave presence/absence, P-R interval, ECG deflection slope and amplitude. These features in combination with clinical data and prior Holter data are used in machine learning models to generate robust rhythm classification tools. These tools are applied on all data collected, leading to highly accurate rhythm classification signals that are provided to and reviewed by clinicians. These systems are developed with the knowledge that they can be improved upon as more clinical data becomes available.

To develop robust arrhythmia detection tools using machine learning techniques, large quantities of data are required. Although there is an abundant amount of traditional Holter (chest-based monitor) data, there is only a minimum quantity of single arm data collected. To meet this requirement, the software may include a generative ECG model to synthesize resemblant single arm data by transposing Holter (chest-based) ECG signals through a GANS machine learning model. The model ingests data from a cardiac monitoring system, Holter data and all major noise sources. In doing so, the tool can generate comparable single arm signal that can be expected to be recorded in the reasonable use of the cardiac monitoring device described herein, including all arrhythmias. Theoretically, all Holter based ECG traces and noise sources can be transposed to the synthesis of the expected signal of which would be collected by a system described herein. This tool can also be used to further expand the system's rhythm classification tools and filtering methods.

The system described herein may further detect and monitor a patient's respiratory rate enabled through an arm-based ECG monitoring system. Traditional ECG monitoring systems, placed on the chest, capture sinusoidal fluctuations in the ECG QRS amplitude as the patients breathe. This is due to the change in the tissue impedance the ECG signal must travel through before reaching the patient's skin while the diaphragm rises and falls. The system described herein follows this same physical approach to detecting a patient's respiratory rate however through a single arm ECG monitoring device. An example of the system can utilize this detected respiratory rate in parallel with a decreasing heart rate for the detection of sleep apnea using a single monitoring device.

The system described herein may be part of a multiple sensor approach to cardiac care. The system described herein may have a 3-axis accelerometer and a 3-axis gyroscope. The sensor may capture the user's motions in parallel with their ECG traces to provide the clinician and patient with a complete understanding of their cardiac health and care plan. This data can be converted to a patient's steps count, and their duration of time in motion, stationary, and lying down. The mobile application may display this information to patients and clinicians to further their personal health goals in parallel with the clinician's cardiac care plans.

The system described herein may further this multiple sensor approach to cardiac care by implementing more sensors including but not limited to; a hemodynamic sensor, skin temperature and color sensor, a galvanic sweat sensor, and SPO2 sensor. The collective information from these sensors can be correlated together to improve the system's filtering methods and rhythm classification tools. An example can utilize the accelerometer and gyroscope data to predict the motion of patients to build an adaptive filter to compensate for the motion artifact expected within the system's ECG signal. Another example can incorporate the pulse wave collected from the pulse oximetry sensor, such that it can be correlated to other hemodynamic signals and further improve the system's heart health reporting.

The majority of the system's core technology resides within the electrical, mechanical and filtering/analytic tools. However, a mobile application and cloud based infrastructure is required to provide a complete end-to-end ECG monitoring solution. The system uses Bluetooth® low-energy communication transfer protocol to transfer collected data from the cardiac monitoring device to the mobile application. The data is then processed, stored and sent to cloud services. The mobile application serves as the user interface to operate the cardiac monitoring device. The cloud services may hold the filtering/analytic tools described above and manage the data for permanent storage. The system also provides the ability for clinics to review the outcomes of a monitoring session through an ECG summary report and provide a diagnosis to the patient.

The software tools utilize a user feedback signal quality index tool and template guided positioning tool. These systems provide real time information to the users of the patient's signal quality and guide the user on the correct placement of the cardiac monitoring device. The signal quality index analyzes the power of noise within different bandpass frequencies in a constrained time segment to judge the general quality of the signal. The template position guiding tool creates a continually updating template (average) ECG beat for the patient during a period of clean data saving these in an array of templated beats. These templated beats are defined as clean beats that represent various positions on the patient's arm and/or of beats of different patient-specific morphologies. Users can use these tools in real time to assist them in collecting high quality ECG signals.

An additional source of error could be that the cardiac monitoring device is completely off, or unattached to, the user. The computing device running the software connected to the cardiac monitoring device may run a software algorithm that uses the recorded signal to determine when the device has been removed by the user. During this time, no ECG is collected, and thus this signal is not processed any further. The algorithm operates by searching for a signal where no QRS is detected and where the system's programmable gain setting is at is maximum sensitivity, which is typical to device off scenarios.

In some instances, in response to detecting the errors, the system may control the plurality of dry electrodes to record a subsequent set of electrocardiogram signals. The system may receive the subsequent set of electrocardiogram signals from the cardiac monitoring device and process a set duration of time (e.g., 3 seconds) within the third set of electrocardiogram signals. The system may output, to a clinical display device (e.g., a device used by a clinician or technologist), the processed third set of electrocardiogram signals over the set duration of time for review by an operator.

This feature may be equivalent to a Live-Look-In tool. As described above, the system processes the collected ECG and an error due to signal quality and poor positioning is alerted. When this error occurs, the system will alert the user and guide them on how to resolve the poor signal quality. If they are unable to do so, this technique is then used. Activated through user action, the device and system connect to stream data in real time from the cardiac monitoring device to the computing device running the software application, then to a server device that outputs the data for display on a GUI for a trained ECG technologist to review the signal. In real time, the technologist is able to view the signal and instruct the user on how to adjust the position of the device to collect a better quality signal.

The Cloud services include a software tool to review, edit and analyze collected ECG signals from patients. This is typical within traditional ECG monitoring solutions. However, traditional tools require clinicians to scan through hours of data searching for irregular patterns within the patient's ECG. This process is often slow and tedious, especially when signal traces are infected with noise. To improve the speed of review, software tools may display an averaged template beat for each region of interest the clinician is reviewing. This templated beat provides a clean image of the patient's majority ECG morphology to visually assist in the removal of noise within the ECG signal. In doing so, clinicians can quickly scan through noisy traces with ease. [Within the current system described herein]

The system described herein may implement within the mobile application a patient's general health feature enabled via an arm based biometric sensing device. The system may report on the general health of the patient, as well as the activity levels, respiration rate, and sleep quality. The device may incorporate sensor information from a multitude of sensors including but not limited to; a hemodynamic sensor, skin temperature and color sensor, a galvanic sweat sensor and SPO2 sensor. One example of the system has all sensors placed on a user's single arm.

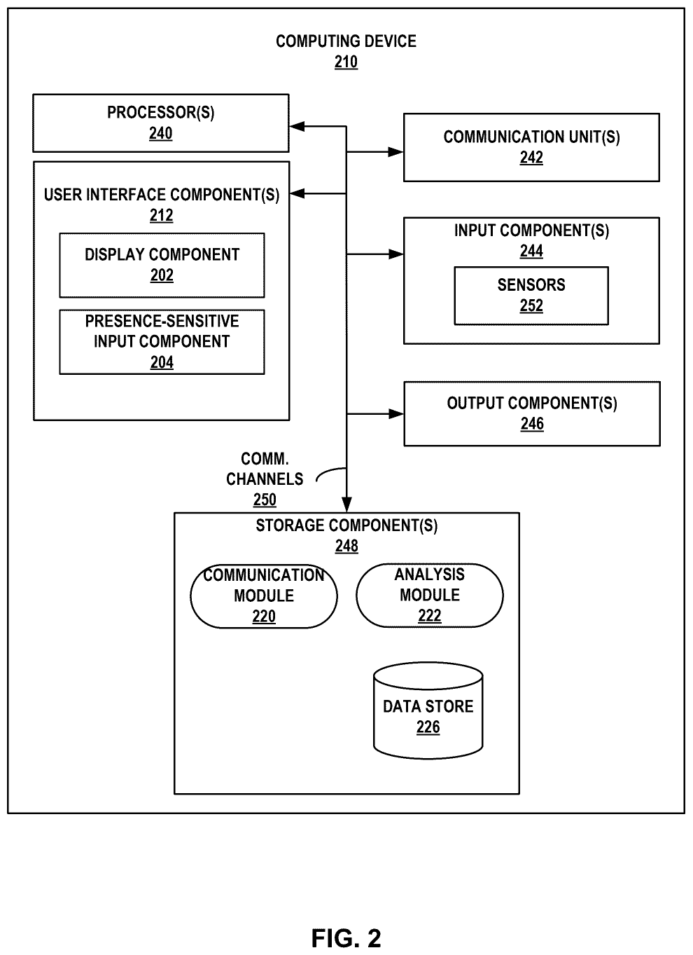

FIG. 2 is a block diagram illustrating a more detailed example of a computing device configured to perform the techniques described herein. Computing device 210 of FIG. 2 is described below as an example of computing device 110 of FIG. 1. FIG. 2 illustrates only one particular example of computing device 210, and many other examples of computing device 210 may be used in other instances and may include a subset of the components included in example computing device 210 or may include additional components not shown in FIG. 2.

Computing device 210 may be any computer with the processing power required to adequately execute the techniques described herein. For instance, computing device 210 may be any one or more of a mobile computing device (e.g., a smartphone, a tablet computer, a laptop computer, etc.), a desktop computer, a smarthome component (e.g., a computerized appliance, a home security system, a control panel for home components, a lighting system, a smart power outlet, etc.), a vehicle, a wearable computing device (e.g., a smart watch, computerized glasses, a heart monitor, a glucose monitor, smart headphones, etc.), a virtual reality/augmented reality/extended reality (VR/AR/XR) system, a video game or streaming system, a transponder, a network modem, router, or server system, or any other computerized device that may be configured to perform the techniques described herein.

As shown in the example of FIG. 2, computing device 210 includes user interface components (UIC) 212, one or more processors 240, one or more communication units 242, one or more input components 244, one or more output components 246, and one or more storage components 248. UIC 212 includes display component 202 and presence-sensitive input component 204. Storage components 248 of computing device 210 include communication module 220, analysis module 222, and data store 226.

One or more processors 240 may implement functionality and/or execute instructions associated with computing device 210 to capture, extract, and analyze electrocardiograms using a cardiac monitoring system. That is, processors 240 may implement functionality and/or execute instructions associated with computing device 210 to detect rhythmic abnormalities from data captured by a cardiac monitoring system described herein.

Examples of processors 240 include any combination of application processors, display controllers, auxiliary processors, one or more sensor hubs, and any other hardware configured to function as a processor, a processing unit, or a processing device, including dedicated graphical processing units (GPUs). Modules 220 and 222 may be operable by processors 240 to perform various actions, operations, or functions of computing device 210. For example, processors 240 of computing device 210 may retrieve and execute instructions stored by storage components 248 that cause processors 240 to perform the operations described with respect to modules 220 and 222. The instructions, when executed by processors 240, may cause computing device 210 capture, extract, and analyze electrocardiograms using a cardiac monitoring system.

Communication module 220 may execute locally (e.g., at processors 240) to provide functions associated with controlling a cardiac monitoring system and receiving and/or transmitting data. In some examples, communication module 220 may act as an interface to a remote service accessible to computing device 210. For example, communication module 220 may be an interface or application programming interface (API) to a remote server that controls a cardiac monitoring system and receives and/or transmits data.

In some examples, analysis module 222 may execute locally (e.g., at processors 240) to provide functions associated with extracting an electrocardiogram from received data and detecting abnormalities. In some examples, analysis module 222 may act as an interface to a remote service accessible to computing device 210. For example, analysis module 222 may be an interface or application programming interface (API) to a remote server that extracts an electrocardiogram from received data and detects abnormalities.

One or more storage components 248 within computing device 210 may store information for processing during operation of computing device 210 (e.g., computing device 210 may store data accessed by modules 220 and 222 during execution at computing device 210). In some examples, storage component 248 is a temporary memory, meaning that a primary purpose of storage component 248 is not long-term storage. Storage components 248 on computing device 210 may be configured for short-term storage of information as volatile memory and therefore not retain stored contents if powered off. Examples of volatile memories include random access memories (RAM), dynamic random access memories (DRAM), static random access memories (SRAM), and other forms of volatile memories known in the art.

Storage components 248, in some examples, also include one or more computer-readable storage media. Storage components 248 in some examples include one or more non-transitory computer-readable storage mediums. Storage components 248 may be configured to store larger amounts of information than typically stored by volatile memory. Storage components 248 may further be configured for long-term storage of information as non-volatile memory space and retain information after power on/off cycles. Examples of non-volatile memories include magnetic hard discs, optical discs, floppy discs, flash memories, or forms of electrically programmable memories (EPROM) or electrically erasable and programmable (EEPROM) memories. Storage components 248 may store program instructions and/or information (e.g., data) associated with modules 220 and 222 and data store 226. Storage components 248 may include a memory configured to store data or other information associated with modules 220 and 222 and data store 226.

Communication channels 250 may interconnect each of the components 212, 240, 242, 244, 246, and 248 for inter-component communications (physically, communicatively, and/or operatively). In some examples, communication channels 250 may include a system bus, a network connection, an inter-process communication data structure, or any other method for communicating data.

One or more communication units 242 of computing device 210 may communicate with external devices via one or more wired and/or wireless networks by transmitting and/or receiving network signals on one or more networks. Examples of communication units 242 include a network interface card (e.g., such as an Ethernet card), an optical transceiver, a radio frequency transceiver, a GPS receiver, a radio-frequency identification (RFID) transceiver, a near-field communication (NFC) transceiver, or any other type of device that can send and/or receive information. Other examples of communication units 242 may include short wave radios, cellular data radios, wireless network radios, as well as universal serial bus (USB) controllers.

One or more input components 244 of computing device 210 may receive input. Examples of input are tactile, audio, and video input. Input components 244 of computing device 210, in one example, include a presence-sensitive input device (e.g., a touch sensitive screen, a PSD), mouse, keyboard, voice responsive system, camera, microphone or any other type of device for detecting input from a human or machine. In some examples, input components 244 may include one or more sensor components (e.g., sensors 252). Sensors 252 may include one or more biometric sensors (e.g., fingerprint sensors, retina scanners, vocal input sensors/microphones, facial recognition sensors, cameras), one or more location sensors (e.g., GPS components, Wi-Fi components, cellular components), one or more temperature sensors, one or more movement sensors (e.g., accelerometers, gyros), one or more pressure sensors (e.g., barometer), one or more ambient light sensors, and one or more other sensors (e.g., infrared proximity sensor, hygrometer sensor, and the like). Other sensors, to name a few other non-limiting examples, may include a radar sensor, a lidar sensor, a sonar sensor, a heart rate sensor, an ECG sensor, magnetometer, glucose sensor, olfactory sensor, compass sensor, or a step counter sensor.

One or more output components 246 of computing device 210 may generate output in a selected modality. Examples of modalities may include a tactile notification, vibration notification, audible notification, visual notification, machine generated voice notification, or other modalities. Output components 246 of computing device 210, in one example, include a presence-sensitive display, a sound card, a video graphics adapter card, a speaker, a cathode ray tube (CRT) monitor, a liquid crystal display (LCD), a light emitting diode (LED) display, an organic LED (OLED) display, a virtual/augmented/extended reality (VR/AR/XR) system, a three-dimensional display, or any other type of device for generating output to a human or machine in a selected modality.

UIC 212 of computing device 210 may include display component 202 and presence-sensitive input component 204. Display component 202 may be a screen, such as any of the displays or systems described with respect to output components 246, at which information (e.g., a visual indication) is displayed by UIC 212 while presence-sensitive input component 204 may detect an object at and/or near display component 202.

While illustrated as an internal component of computing device 210, UIC 212 may also represent an external component that shares a data path with computing device 210 for transmitting and/or receiving input and output. For instance, in one example, UIC 212 represents a built-in component of computing device 210 located within and physically connected to the external packaging of computing device 210 (e.g., a screen on a mobile phone). In another example, UIC 212 represents an external component of computing device 210 located outside and physically separated from the packaging or housing of computing device 210 (e.g., a monitor, a projector, etc. that shares a wired and/or wireless data path with computing device 210).

UIC 212 of computing device 210 may detect two-dimensional and/or three-dimensional gestures as input from a user of computing device 210. For instance, a sensor of UIC 212 may detect a user's movement (e.g., moving a hand, an arm, a pen, a stylus, a tactile object, etc.) within a threshold distance of the sensor of UIC 212. UIC 212 may determine a two or three-dimensional vector representation of the movement and correlate the vector representation to a gesture input (e.g., a hand-wave, a pinch, a clap, a pen stroke, etc.) that has multiple dimensions. In other words, UIC 212 can detect a multi-dimension gesture without requiring the user to gesture at or near a screen or surface at which UIC 212 outputs information for display. Instead, UIC 212 can detect a multi-dimensional gesture performed at or near a sensor which may or may not be located near the screen or surface at which UIC 212 outputs information for display.

In accordance with the techniques of this disclosure, a cardiac monitoring system includes computing device 210 and a monitoring device comprising a plurality of electrodes. Computing device 210 and the monitoring device may be integrated into a same physical device or separate devices in which wired or wireless communication with one another occur. In some instances, the monitoring device also includes a strap, the strap being one or more of a hook-and-loop strap, a buckle clip strap, a belt strap, and an elastic loop.

When the monitoring device is placed around an extremity of a patient, communication module 220 controls the plurality of electrodes to record one or more electrocardiogram signals. The one or more electrocardiogram signals may be one or more clinically relevant electrocardiogram signals. In some instances, the extremity of the patient may be an arm of the patient or a leg of the patient.

In some instances, computing device 210 may be one of a mobile computing device, a cloud-based server, or a desktop computing device. In other instances, computing device 210 may be a distributed computing system comprising two or more of a mobile computing device, a cloud-based server, or a desktop computing device.

In some instances, the plurality of electrodes of the monitoring device may be a plurality of dry electrodes that, when placed around the extremity of the patient, do not require conductive gel and do not require adhesive to record the one or more electrocardiogram signals. Additionally or alternatively, each of the plurality of electrodes may include a high-sensitivity analog front end circuit. In some such instances, each of the high-sensitivity analog front end circuits may be one or more of a right leg drive (RLD) feedback system and a direct current (DC) biasing reduction system. While the plurality of electrodes is recording the one or more electrocardiogram signals, the high-sensitivity analog front end circuits may be configured to reduce noise in the one or more electrocardiogram signals, wherein the one or more data packets are indicative of the one or more electrocardiogram signals after noise reduction.

In some such instances, the high-sensitivity analog front end circuits being configured to reduce the noise in the one or more electrocardiogram signals may include the high-sensitivity analog front end circuits being configured to amplify the one or more electrocardiogram signals and apply one or more of an RLD feedback system, a DC biasing reduction system, and one or more fixed and/or adaptive analog filters to the one or more electrocardiogram signals to reduce the noise in the one or more electrocardiogram signals. While fixed and adaptive analog filters may have different cutoff frequency parameters, communication module 220 may adjust the cutoff frequency of an adaptive analog filter digitally depending on the noise recorded by the system.

Communication module 220 may receive, from the monitoring device, one or more data packets, the one or more data packets indicative of the one or more electrocardiogram signals. Analysis module 222 may apply one or more adaptive software filters to the one or more data packets to extract an electrocardiogram for the patient.

In some instances, analysis module 222 may further analyze the electrocardiogram of the patient. Analysis module 222 may be configured to detect one or more rhythm abnormalities in the electrocardiogram from that analysis. In some such instances, each of the one or more rhythm abnormalities may corresponds to a section of the electrocardiogram, where analysis module 222 may generate a graphical user interface that presents at least the distinct, non-consecutive QRS wave sections of the electrocardiogram,

In some instances, analysis module 222 may generate a graphical user interface that includes an indication of the electrocardiogram. Communication module 220 may output, for display on a display device, the graphical user interface.

In some instances, communication module 220 may receive, from one or more ancillary devices, activity information. The activity information may include any one or more of a daily step count, an activity level, a sleep pattern, a blood pressure, a pulse oxygenation level, a carbon dioxide level, a detected fall, sleep apnea detection, a hemodynamic measurement, a skin color, a skin temperature, a sweat detection, and posture detection. Analysis module 222 may generate a graphical user interface that includes an indication of the electrocardiogram and an indication of at least a portion of the activity information, and communication module 220 may output, for display on a display device, the graphical user interface.

In some instances, analysis module 222 may monitor one or more biometrics of the user, the one or more biometrics including any one or more of a blood pressure for the user, fall detection, a respiratory rate for the user, a measure of hemodynamic flow for the user, skin pigmentation level of the user, a skin temperature for the user, and galvanic sweat levels for the user. Analysis module 222 may further generate a graphical user interface that includes an indication of the one or more biometrics and the electrocardiogram, and communication module 220 may output, for display on a display device, the graphical user interface.

In some instances, analysis module 222 may determine one or more of a respiratory rate for the user, a heart rate for the user, and motion of the user. Analysis module 222 may combine one or more of the respiratory rate, the heart rate, and the motion during sleep (e.g., while the patient is sleeping) to detect sleep apnea.

In some instances, analysis module 222 may analyze the electrocardiogram of the patient. Analysis module 222 may diagnose a condition in the patient based on the electrocardiogram.

In some instances, communication module 220 may receive an indication of user input comprising a human annotation of detected rhythm abnormalities to generate an annotated electrocardiogram. Communication module 220 may transmit the annotated electrocardiogram to a computing device associated with a prescribing physician.

In some instances, communication module 220 and analysis module 222 may repeat actions to extract a plurality of electrocardiograms for the patient, each cardiogram being associated with a unique time span for the patient. Analysis module 222 may determine one or more of a frequency and a pattern of arrhythmias across the plurality of electrocardiograms for the patient.

In accordance with the techniques of this disclosure, analysis module 222 may analyze a plurality of electrocardiograms for a patient, each electrocardiogram of the plurality of electrocardiograms being recorded at unique times. Analysis module 222 may determine that a subset of electrocardiograms from the plurality of electrocardiograms include a rhythmic abnormality. Analysis module 222 may generate a graphical user interface that includes at least a first grouping of graphical indications and a second grouping of one or more graphical indications. Each graphical indication in the first grouping may represent an electrocardiogram from the subset of electrocardiograms that include the rhythmic abnormality, and each graphical indication in the second grouping may represent an electrocardiogram from the plurality of electrocardiograms that is not included in the subset of electrocardiograms. Communication module 220 may output, for display on a display device, the graphical user interface.

In some instances, analysis module 222 may generate the graphical user interface to include an overlay of an averaged template beat for the patient with each graphical indication in the first grouping.

In some instances, analysis module 222 may determine a diagnostic recommendation based on the subset of electrocardiograms that include a rhythmic abnormality. In such instances, analysis module 222 may generate the graphical user interface to further include a graphical indication of the diagnostic recommendation.

In some instances, analysis module 222 may automatically generate one or more clinical notes based on the subset of electrocardiograms that include a rhythmic abnormality. In such instances, analysis module 222 may generate the graphical user interface to further include a graphical indication of the one or more clinical notes.

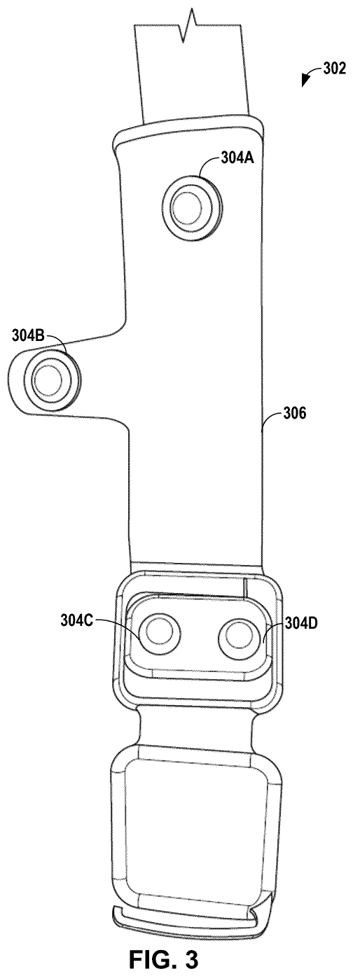

FIG. 3 is a diagram illustrating an example cardiac monitoring device 302, in accordance with the techniques of this disclosure. In the example of FIG. 3, a bicep-worn strap 306 with four metallic electrodes 304A-304D to record clinically-relevant high-definition ECG signals is shown. Cardiac monitoring device 302 may be similar to cardiac monitoring device 102 of FIG. 1 and may perform similar functions as cardiac monitoring device 102. Similarly, electrodes 304A-304D may be similar to electrodes 104A-104C of FIG. 1 and may perform similar functions as electrodes 104A-104C, and strap 306 may be similar to strap 106 of FIG. 1 and may perform similar functions as strap 106.

This dry-electrode solution is enabled by a proprietary high sensitivity analog front end circuit which has been designed to extract the highly attenuated signals collected by the system. The circuit refines its performance by implementing two noise reduction systems-a Right Leg Drive (RLD) feedback system (to remove common mode noise) and a DC biasing reduction system to remove the unwanted dc offset swing. Collecting ECG signals using dry electrodes placed on the user's arm poses other challenges that may be overcome with the use of aggressive hardware and software filters. High order traditional hardware filters are utilized along with a suite of adaptive software filters that work to extract a clinically relevant ECG specific to the system's application.

The electronic component of the system hardware is designed to be reusable, with modest refurbishing required between patient deployments.

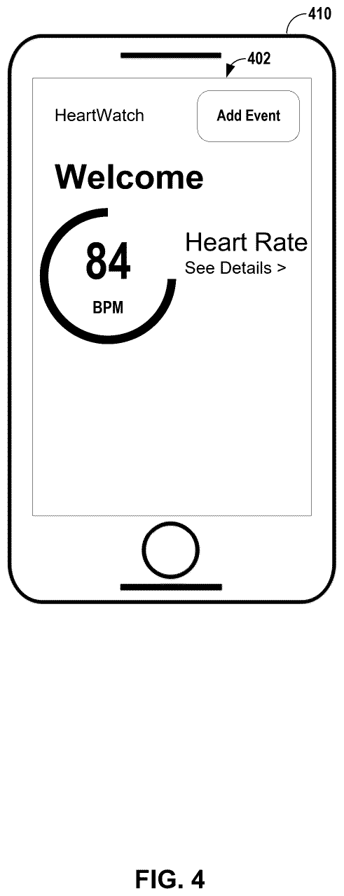

FIG. 4 is an example user interface 402 on a client device 410 showing data from electrocardiograms, in accordance with the techniques of this disclosure. Client device 410 may be similar to computing device 110 of FIG. 1 and/or computing device 210 of FIG. 2 and may perform similar functions as either computing device 110 and/or computing device 210. A phone-based mobile application to facilitate the transfer of the patient's ECG data to a secure cloud server. The mobile application can also, in certain examples, display ancillary information to the patients, such as their daily step count, activity level, and sleep patterns.

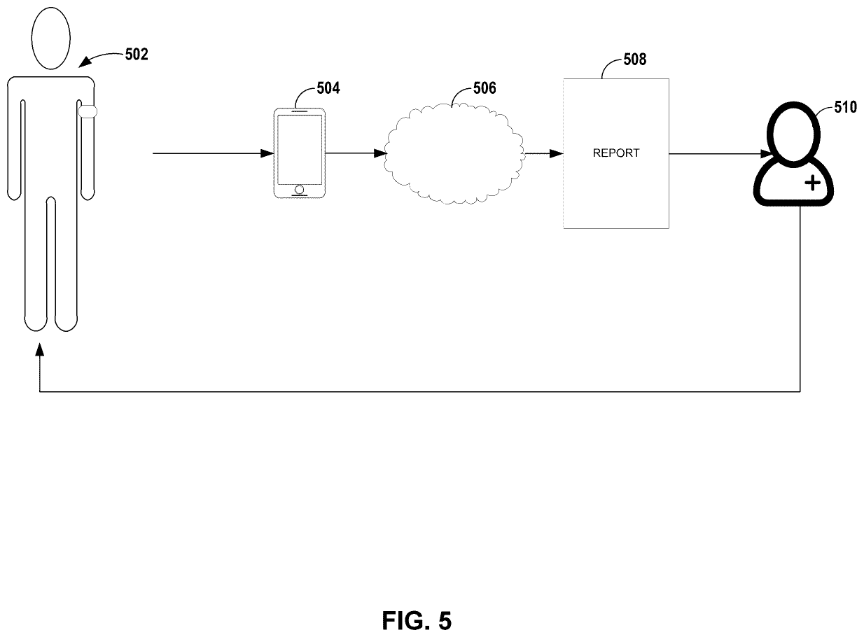

FIG. 5 is an example flow diagram illustrating measuring electrocardiogram signals from a cardiac monitoring device worn by a patient (object 502) and transmitting this data to a certified technologist 510, in accordance with the techniques of this disclosure. Data from a cardiac monitoring device worn by a patient (collectively object 502) may be sent to a mobile device 504 locally. Mobile device 504 may upload the data to a cloud-based system 506. The cloud-based system 506 may include a cloud-based algorithm stack to process the ECG signals and make first-pass arrhythmia classifications and diagnoses, which is placed in report 508. The first-pass data in report 508 may be sent to desktop software to facilitate human annotation of detected rhythm abnormalities by certified cardiac technologists 510. The software may enable these technologists to easily create rhythm reports to be view by the prescribing physician.

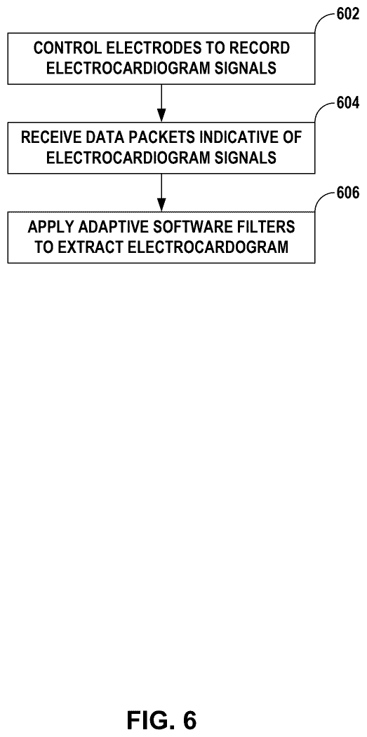

FIG. 6 is a flow chart illustrating an example mode of operation. The techniques of FIG. 6 may be performed by one or more processors of a computing device, such as system 100 of FIG. 1 and/or computing device 210 illustrated in FIG. 2. For purposes of illustration only, the techniques of FIG. 6 are described within the context of computing device 210 of FIG. 2, although computing devices having configurations different than that of computing device 210 may perform the techniques of FIG. 6.

In accordance with the techniques of this disclosure, communication module 220 may control a plurality of dry electrodes on a cardiac monitoring device to record one or more electrocardiogram signals when placed around an extremity of a patient (602). Communication module 220 may receive the one or more electrocardiogram signals from the cardiac monitoring device (604). Analysis module 222 may apply one or more adaptive software filters to the one or more data packets to extract an electrocardiogram for the patient (606).

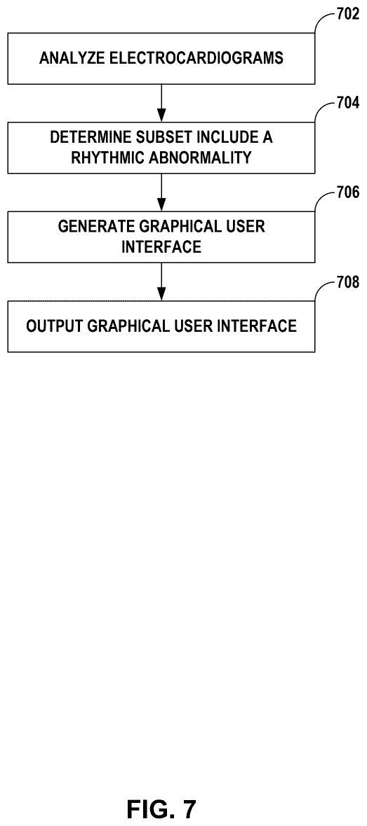

FIG. 7 is a flow chart illustrating an example mode of operation. The techniques of FIG. 7 may be performed by one or more processors of a computing device, such as system 100 of FIG. 1 and/or computing device 210 illustrated in FIG. 2. For purposes of illustration only, the techniques of FIG. 7 are described within the context of computing device 210 of FIG. 2, although computing devices having configurations different than that of computing device 210 may perform the techniques of FIG. 7.

In accordance with the techniques of this disclosure, analysis module 222 may analyze a plurality of electrocardiograms for a patient, each electrocardiogram of the plurality of electrocardiograms being recorded at unique times (702). Analysis module 222 may determine that a subset of electrocardiograms from the plurality of electrocardiograms include a rhythmic abnormality (704). Analysis module 222 may generate a graphical user interface that includes at least a first grouping of graphical indications and a second grouping of one or more graphical indications (706), each graphical indication in the first grouping representing an electrocardiogram from the subset of electrocardiograms that include the rhythmic abnormality, and each graphical indication in the second grouping representing an electrocardiogram from the plurality of electrocardiograms that is not included in the subset of electrocardiograms. Communication module 220 may output, for display on a display device, the graphical user interface (708).

Although the various examples have been described with reference to preferred implementations, persons skilled in the art will recognize that changes may be made in form and detail without departing from the spirit and scope thereof.

It is to be recognized that depending on the example, certain acts or events of any of the techniques described herein can be performed in a different sequence, may be added, merged, or left out altogether (e.g., not all described acts or events are necessary for the practice of the techniques). Moreover, in certain examples, acts or events may be performed concurrently, e.g., through multi-threaded processing, interrupt processing, or multiple processors, rather than sequentially.

In one or more examples, the functions described may be implemented in hardware, software, firmware, or any combination thereof. If implemented in software, the functions may be stored on or transmitted over as one or more instructions or code on a computer-readable medium and executed by a hardware-based processing unit. Computer-readable media may include computer-readable storage media, which corresponds to a tangible medium such as data storage media, or communication media including any medium that facilitates transfer of a computer program from one place to another, e.g., according to a communication protocol. In this manner, computer-readable media generally may correspond to (1) tangible computer-readable storage media which is non-transitory or (2) a communication medium such as a signal or carrier wave. Data storage media may be any available media that can be accessed by one or more computers or one or more processors to retrieve instructions, code and/or data structures for implementation of the techniques described in this disclosure. A computer program product may include a computer-readable medium.

It is contemplated that the various aspects, features, processes, and operations from the various embodiments may be used in any of the other embodiments unless expressly stated to the contrary. Certain operations illustrated may be implemented by a computer executing a computer program product on a non-transient, computer-readable storage medium, where the computer program product includes instructions causing the computer to execute one or more of the operations, or to issue commands to other devices to execute one or more operations.

By way of example, and not limitation, such computer-readable storage media can comprise RAM, ROM, EEPROM, CD-ROM or other optical disk storage, magnetic disk storage, or other magnetic storage devices, flash memory, or any other medium that can be used to store desired program code in the form of instructions or data structures and that can be accessed by a computer. Also, any connection is properly termed a computer-readable medium. For example, if instructions are transmitted from a website, server, or other remote source using a coaxial cable, fiber optic cable, twisted pair, digital subscriber line (DSL), or wireless technologies such as infrared, radio, and microwave, then the coaxial cable, fiber optic cable, twisted pair, DSL, or wireless technologies such as infrared, radio, and microwave are included in the definition of medium. It should be understood, however, that computer-readable storage media and data storage media do not include connections, carrier waves, signals, or other transitory media, but are instead directed to non-transitory, tangible storage media. Disk and disc, as used herein, includes compact disc (CD), laser disc, optical disc, digital versatile disc (DVD), floppy disk and Blu-ray disc, where disks usually reproduce data magnetically, while discs reproduce data optically with lasers. Combinations of the above should also be included within the scope of computer-readable media.

Instructions may be executed by one or more processors, such as one or more digital signal processors (DSPs), general purpose microprocessors, application specific integrated circuits (ASICs), field programmable logic arrays (FPGAs), or other equivalent integrated or discrete logic circuitry. Accordingly, the term “processor,” as used herein may refer to any of the foregoing structures or any other structure suitable for implementation of the techniques described herein. In addition, in some aspects, the functionality described herein may be provided within dedicated hardware and/or software modules configured for encoding and decoding, or incorporated in a combined codec. Also, the techniques could be fully implemented in one or more circuits or logic elements.

The techniques of this disclosure may be implemented in a wide variety of devices or apparatuses, including a wireless handset, an integrated circuit (IC) or a set of ICs (e.g., a chip set). Various components, modules, or units are described in this disclosure to emphasize functional aspects of devices configured to perform the disclosed techniques, but do not necessarily require realization by different hardware units. Rather, as described above, various units may be combined in a codec hardware unit or provided by a collection of interoperative hardware units, including one or more processors as described above, in conjunction with suitable software and/or firmware.

Various embodiments of the invention may be implemented at least in part in any conventional computer programming language. For example, some embodiments may be implemented in a procedural programming language (e.g., “C”), or in an object oriented programming language (e.g., “C++”). Other embodiments of the invention may be implemented as a pre-configured, stand-alone hardware element and/or as preprogrammed hardware elements (e.g., application specific integrated circuits, FPGAs, and digital signal processors), or other related components.

Those skilled in the art should appreciate that such computer instructions can be written in a number of programming languages for use with many computer architectures or operating systems. Furthermore, such instructions may be stored in any memory device, such as semiconductor, magnetic, optical or other memory devices, and may be transmitted using any communications technology, such as optical, infrared, microwave, or other transmission technologies.

Among other ways, such a computer program product may be distributed as a removable medium with accompanying printed or electronic documentation (e.g., shrink wrapped software), preloaded with a computer system (e.g., on system ROM or fixed disk), or distributed from a server or electronic bulletin board over the network (e.g., the Internet or World Wide Web). In fact, some embodiments may be implemented in a software-as-a-service model (“SAAS”) or cloud computing model. Of course, some embodiments of the invention may be implemented as a combination of both software (e.g., a computer program product) and hardware. Still other embodiments of the invention are implemented as entirely hardware, or entirely software.

While the various systems described above are separate implementations, any of the individual components, mechanisms, or devices, and related features and functionality, within the various system embodiments described in detail above can be incorporated into any of the other system embodiments herein.

The terms “about” and “substantially,” as used herein, refers to variation that can occur (including in numerical quantity or structure), for example, through typical measuring techniques and equipment, with respect to any quantifiable variable, including, but not limited to, mass, volume, time, distance, wave length, frequency, voltage, current, and electromagnetic field. Further, there is certain inadvertent error and variation in the real world that is likely through differences in the manufacture, source, or precision of the components used to make the various components or carry out the methods and the like. The terms “about” and “substantially” also encompass these variations. The term “about” and “substantially” can include any variation of 5% or 10%, or any amount-including any integer-between 0% and 10%. Further, whether or not modified by the term “about” or “substantially,” the claims include equivalents to the quantities or amounts.

Numeric ranges recited within the specification are inclusive of the numbers defining the range and include each integer within the defined range. Throughout this disclosure, various aspects of this disclosure are presented in a range format. It should be understood that the description in range format is merely for convenience and brevity and should not be construed as an inflexible limitation on the scope of the disclosure. Accordingly, the description of a range should be considered to have specifically disclosed all the possible sub-ranges, fractions, and individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 3, 4, 5, and 6, and decimals and fractions, for example, 1.2, 3.8, 1½, and 4¾ This applies regardless of the breadth of the range. Although the various embodiments have been described with reference to preferred implementations, persons skilled in the art will recognize that changes may be made in form and detail without departing from the spirit and scope thereof.

Various examples of the disclosure have been described. Any combination of the described systems, operations, or functions is contemplated. These and other examples are within the scope of the following claims.

What is claimed is:

1. A cardiac monitoring system comprising:
(a) a monitoring device comprising a plurality of electrodes; and
(b) one or more processors configured to:
(i) when the monitoring device is placed around an extremity of a patient, control the plurality of electrodes to record one or more electrocardiogram signals;
(ii) receive, from the monitoring device, one or more data packets, the one or more data packets indicative of the one or more electrocardiogram signals; and
(iii) apply one or more adaptive software filters to the one or more data packets to extract an electrocardiogram for the patient.

2. The cardiac monitoring system of claim 1, wherein the plurality of electrodes of the monitoring device comprises a plurality of dry electrodes that, when placed around the extremity of the patient, do not require conductive gel and do not require adhesive to record the one or more electrocardiogram signals.

3. The cardiac monitoring system of claim 1, wherein each of the plurality of electrodes comprises a high-sensitivity analog front end circuit.

4. The cardiac monitoring system of claim 3, wherein each of the high-sensitivity analog front end circuits comprises one or more of a right leg drive (RLD) feedback system and a direct current (DC) biasing reduction system.

5. The cardiac monitoring system of claim 3, wherein, while the plurality of electrodes are recording the one or more electrocardiogram signals, the high-sensitivity analog front end circuits are configured to reduce noise in the one or more electrocardiogram signals, wherein the one or more data packets are indicative of the one or more electrocardiogram signals after noise reduction.

6. The cardiac monitoring system of claim 5, wherein the high-sensitivity analog front end circuits being configured to reduce the noise in the one or more electrocardiogram signals comprises the high-sensitivity analog front end circuits being configured to:
(i) amplify the one or more electrocardiogram signals; and
(ii) apply one or more of an RLD feedback system, a DC biasing reduction system, and one or more analog filters to the one or more electrocardiogram signals to reduce the noise in the one or more electrocardiogram signals, wherein the one or more analog filters are either fixed analog filters, adaptive analog filters, or a combination of fixed and adaptive analog filters.

7. The cardiac monitoring system of claim 1, wherein the one or more processors are further configured to:
(i) analyze the electrocardiogram of the patient; and
(ii) detect one or more rhythm abnormalities in the electrocardiogram.

8. The cardiac monitoring system of claim 7, wherein each of the one or more rhythm abnormalities corresponds to a section of the electrocardiogram, wherein the one or more processors are further configured to generate a graphical user interface that presents at least the distinct, non-consecutive QRS wave sections of the electrocardiogram.

9. The cardiac monitoring system of claim 1, wherein the one or more processors are further configured to:
(i) generate a graphical user interface that includes an indication of the electrocardiogram; and
(ii) output, for display on a display device, the graphical user interface.

10. The cardiac monitoring system of claim 1, wherein the one or more processors are further configured to:
(i) receive, from one or more ancillary devices, activity information;
(ii) generate a graphical user interface that includes an indication of the electrocardiogram and an indication of at least a portion of the activity information; and
(iii) output, for display on a display device, the graphical user interface.

11. The cardiac monitoring system of claim 10, wherein the activity information comprises one or more of a daily step count, an activity level, a sleep pattern, a blood pressure, a pulse oxygenation level, a carbon dioxide level, a detected fall, sleep apnea detection, a hemodynamic measurement, a skin color, a skin temperature, a sweat detection, and posture detection.

12. The cardiac monitoring system of any one or more claim 1, wherein the one or more processors are further configured to:
(i) monitor one or more biometrics of the user, the one or more biometrics comprising one or more of a blood pressure for the user, fall detection, a respiratory rate for the user, a measure of hemodynamic flow for the user, skin pigmentation level of the user, a skin temperature for the user, and galvanic sweat levels for the user;
(ii) generate a graphical user interface that includes an indication of the one or more biometrics and the electrocardiogram; and
(iii) output, for display on a display device, the graphical user interface.

13. The cardiac monitoring system of any one or more claim 1, wherein the one or more processors are further configured to:
(i) determine one or more of a respiratory rate for the user, a heart rate for the user, and motion of the user; and
(ii) combine one or more of the respiratory rate, the heart rate, and the motion during sleep to detect sleep apnea.

14. The cardiac monitoring system of claim 1, wherein the one or more processors are part of a computing device, wherein the computing device comprises one of a mobile computing device, a cloud-based server, or a desktop computing device.

15. The cardiac monitoring system of claim 1, wherein the one or more processors are part of a computing device, wherein the computing device comprises a distributed computing system comprising two or more of a mobile computing device, a cloud-based server, or a desktop computing device.

16. The cardiac monitoring system of claim 1, wherein the one or more processors are further configured to:
(i) analyze the electrocardiogram of the patient; and
(ii) diagnose a condition in the patient based on the electrocardiogram.

17. The cardiac monitoring system of claim 1, wherein the one or more processors are further configured to:
(i) receive an indication of user input comprising a human annotation of detected rhythm abnormalities to generate an annotated electrocardiogram; and
(ii) transmit the annotated electrocardiogram to a computing device associated with a prescribing physician.

18. The cardiac monitoring system of claim 1, wherein the one or more processors are further configured to:
(i) repeat the steps in claim 1 to extract a plurality of electrocardiograms for the patient, each cardiogram being associated with a unique time span for the patient; and
(i) determine one or more of a frequency and a pattern of arrhythmias across the plurality of electrocardiograms for the patient.

19. The cardiac monitoring system of claim 1, wherein the monitoring device comprises a strap, the strap comprises one or more of:
(a) a hook-and-loop strap,
(b) a buckle clip strap,
(c) a belt strap,
(d) an elastic loop, and
(e) an elastomer strap with one or more of the plurality of electrodes embedded within the elastomer strap.

20. The cardiac monitoring system of claim 1, wherein the extremity of the patient comprises an arm of the patient or a leg of the patient.

21. The cardiac monitoring system of claim 1, wherein the one or more electrocardiogram signals comprise one or more clinically relevant electrocardiogram signals.

22. A cardiac monitoring device comprising:
(a) a self-adhering adjustable length strap; and
(b) a plurality of electrodes configured to record one or more electrocardiogram signals when the cardiac monitoring device is placed around an extremity of a patient,
wherein the cardiac monitoring device, when placed around the extremity of the patient, does not require conductive gel and does not require adhesive to record the one or more electrocardiogram signals.

23. The cardiac monitoring device of claim 22, wherein each of the plurality of electrodes comprises a high-sensitivity analog front end circuit.

24. The cardiac monitoring device of claim 23, wherein each of the high-sensitivity analog front end circuits comprises one or more of a common mode noise reduction system, a right leg drive (RLD) feedback system, and a direct current (DC) biasing reduction system.

25. The cardiac monitoring device of claim 22, wherein, while the plurality of electrodes are recording the one or more electrocardiogram signals, the high-sensitivity analog front end circuits are configured to reduce noise in the one or more electrocardiogram signals, wherein the one or more data packets are indicative of the one or more electrocardiogram signals after noise reduction.

26. The cardiac monitoring device of claim 22, wherein the strap comprises one or more of:
(a) a hook-and-loop strap,
(b) a buckle clip strap,
(c) a belt strap,
(d) an elastic loop, and
(e) an elastomer strap with one or more of the plurality of electrodes embedded within the elastomer strap.

27. The cardiac monitoring device of claim 22, wherein the extremity of the patient comprises an arm of the patient or a leg of the patient.

28. A method comprising:
(i) controlling, by one or more processors, a plurality of dry electrodes on a cardiac monitoring device to record one or more electrocardiogram signals when placed around an extremity of a patient;
(ii) receiving, by the one or more processors, the one or more electrocardiogram signals from the cardiac monitoring device; and
(iii) apply one or more adaptive software filters to the one or more data packets to extract an electrocardiogram for the patient.

29. The method of claim 28, further comprising training an electrocardiogram model, the electrocardiogram model comprising a plurality of patient specific templates on prior clean data.

30. The method of claim 29, wherein the plurality of patient specific templates each comprise average PQRST models.

31. The method of claim 28, wherein applying the one or more adaptive filters comprises applying the electrocardiogram model to the one or more electrocardiogram signals to remove one or more distortions in the one or more electrocardiogram signals to extract the electrocardiogram for the patient.

32. The method of claim 28, wherein the one or more electrocardiogram signals comprise a first set of electrocardiogram signals, and wherein the method further comprises:
(i) controlling, by the one or more processors, the plurality of dry electrodes to record a second set of electrocardiogram signals;
(ii) receiving, by the one or more processors, the second set of electrocardiogram signals from the cardiac monitoring device;
(iii) analyzing, by the one or more processors, the second set of electrocardiogram signals;
(iv) determining, by the one or more processors, an error in the recording of the second set of electrocardiogram signals; and
(v) outputting, by the one or more processors, an alert indicating the error in the recording of the second set of electrocardiogram signals.

33. The method of claim 32, wherein the error in the recording of the second set of electrocardiogram signals comprises one or more of a poor signal quality, a device-off-body detection error, and a poor positioning of the cardiac monitoring device.

34. The method of claim 33, wherein the error comprises the poor positioning of the cardiac monitoring device, wherein the alert comprises a template guided positioning tool.

35. The method of claim 32, further comprising, in response to determining the error:
(i) controlling, by the one or more processors, the plurality of dry electrodes to record a third set of electrocardiogram signals;
(ii) receiving, by the one or more processors, the third set of electrocardiogram signals from the cardiac monitoring device;
(iii) processing, by the one or more processors, a set duration of time within the third set of electrocardiogram signals;
(iv) outputting, by the one or more processors and to a clinical display device, the processed third set of electrocardiogram signals over the set duration of time for review by an operator.

36. The method of claim 28, wherein the one or more adaptive filters comprise one or more of an initial pass rhythm classification tool, a QRS detection algorithm, a peak finder detection algorithm, a physiological constraint model, and a patient specific template.

37. A method comprising:
(i) analyzing, by one or more processors, a plurality of electrocardiograms for a patient, each electrocardiogram of the plurality of electrocardiograms being recorded at unique times;
(ii) determining, by the one or more processors, that a subset of electrocardiograms from the plurality of electrocardiograms include a rhythmic abnormality;
(iii) generating, by the one or more processors, a graphical user interface that includes at least a first grouping of graphical indications and a second grouping of one or more graphical indications, each graphical indication in the first grouping representing an electrocardiogram from the subset of electrocardiograms that include the rhythmic abnormality, and each graphical indication in the second grouping representing an electrocardiogram from the plurality of electrocardiograms that is not included in the subset of electrocardiograms; and
(iv) outputting, by the one or more processors and for display on a display device, the graphical user interface.

38. The method of claim 37, further comprising generating, by the one or more processors, the graphical user interface to include an overlay of an averaged template beat for the patient with each graphical indication in the first grouping.

39. The method of claim 37, further comprising:
(i) determining, by the one or more processors, a diagnostic recommendation based on the subset of electrocardiograms that include a rhythmic abnormality; and
(ii) generating, by the one or more processors, the graphical user interface to further include a graphical indication of the diagnostic recommendation.

40. The method of claim 37, further comprising:
(i) automatically generating, by the one or more processors, one or more clinical notes based on the subset of electrocardiograms that include a rhythmic abnormality; and
(ii) generating, by the one or more processors, the graphical user interface to further include a graphical indication of the one or more clinical notes.
